(19) United States
(12) Patent Application Publication
(10) Pub. No.: US 2025/0331794 A1
(43) Pub. Date: Oct. 30, 2025

(54) RADIOGRAPHIC IMAGING SUPPORT APPARATUS, RADIOGRAPHIC IMAGING SUPPORT SYSTEM, RADIOGRAPHIC IMAGING SUPPORT METHOD, AND STORAGE MEDIUM

(71) Applicant: Konica Minolta, Inc., Tokyo (JP)

(72) Inventor: Mizuki YOSHIDA, Tokyo (JP)

(21) Appl. No.: 19/184,070

(22) Filed: Apr. 21, 2025

(30) Foreign Application Priority Data
Apr. 25, 2024 (JP) 2024-071193

(51) Int. Cl. A61B 6/46; A61B 6/00

(52) U.S. Cl. CPC A61B 6/461; A61B 6/4405

(57) ABSTRACT

A radiographic imaging support apparatus includes a hardware processor and a display. The hardware processor obtains an optical image that shows a portable radiographic imaging device and a subject. The hardware processor determines an imaging range, based on examination information related to an examination of the subject. The display displays information for aligning a position of the radiographic imaging device in the obtained optical image and a position of the determined imaging range.

### CROSS-REFERENCE TO RELATED APPLICATIONS

The entire disclosure of Japanese Patent Application No. 2024-071193 filed on Apr. 25, 2024, is incorporated herein by reference in its entirety.

### BACKGROUND OF THE INVENTION

### Technical Field

The present invention relates to a radiographic imaging support apparatus, a radiographic imaging support system, a radiographic imaging support method, and a storage medium.

### Description of Related Art

In radiography, whether an appropriate radiographic image can be obtained depends on the positional relation among a patient, a tube, and a cassette-type imaging device. In a case where an imaging device is fixed to an imaging stand in performing imaging and the position of the tube is linked to the position of the imaging stand, the position of the tube is linked to the position of the imaging device. Therefore, only the position of the patient needs to be considered. However, in cassette imaging in which the imaging device is not fixed to the imaging stand, a radiologist manually adjusts the entire positional relationship among the patient, the tube, and the imaging device. If one of the patient, the tube, and the imaging device is shifted, an appropriate radiographic image cannot be obtained. Accordingly, re-imaging may be required.

Japanese Unexamined Patent Publication No. 2000-23955 describes a display function of displaying the distance between the center of a radiation source and the center of an imaging means as relative positions thereof. Japanese Unexamined Patent Publication No. 2019-33830 describes changing the display position of a positioning indicator image that indicates the preset position of a subject, in accordance with a change in the position of the cassette in a camera image. Japanese Unexamined Patent Publication No. 2009-050693 describes a technology of notifying whether the distance between the radiation source and the radiation detector calculated based on signals transmitted from the radiation detector is equal to the SID (source to image receptor distance) at the time of radiographic imaging.

In cassette imaging, the patient, the tube, and the imaging device need to be in a correct positional relationship; and both the distance between the tube and the imaging device and the angle of the imaging device to the radiation need to be appropriately set, depending on the imaging part of the subject. With conventional technology, it is possible to display information on the distance and the angle between the tube and the imaging device. However, it is not possible to check whether both of (i) the distance between the tube and the imaging device and (ii) the angle of the imaging device to the radiation are appropriate.

To solve the above-described problem, an object of the present invention is to provide a radiographic imaging support apparatus, a radiographic imaging support system, a radiographic imaging support method, and a storage medium that allow confirmation on whether the positional relationship between the radiation source and the imaging device is appropriate.

### SUMMARY OF THE INVENTION

In order to solve the above problem, according to an aspect of the present invention, there is provided a radiographic imaging support apparatus including a hardware processor and a display, wherein: the hardware processor obtains an optical image that shows a portable radiographic imaging device and a subject, the hardware processor determines an imaging range, based on examination information related to an examination of the subject, and the display displays information for aligning a position of the radiographic imaging device in the obtained optical image and a position of the determined imaging range.

According to an aspect of the present invention, there is provided a radiographic imaging support method including: obtaining an optical image that shows a portable radiographic imaging device and a subject; determining an imaging range, based on examination information related to an examination of the subject; and displaying information for aligning a position of the radiographic imaging device in the obtained optical image and a position of the determined imaging range.

According to an aspect of the present invention, there is provided a non-transitory computer-readable storage medium storing a program for causing a computer to: obtain an optical image that shows a portable radiographic imaging device and a subject, determine an imaging range, based on examination information related to an examination of the subject, and display information for aligning a position of the radiographic imaging device in the obtained optical image and a position of the determined imaging range.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic configuration of a radiographic imaging support system according to a first embodiment;

FIG. 2 is a block diagram of an imaging device according to the first embodiment;

FIG. 3 is an example of a marker section on the imaging device according to the first embodiment;

FIG. 4 is a block diagram of an imaging support system server according to the first embodiment;

FIG. 5 is a flowchart as an example of operations of the imaging support system server that determines whether the relative position between the radiation source and the imaging device is appropriate before radiographic imaging according to the first embodiment;

FIG. 6 illustrates the relation in dimensions between the imaging device arranged at an appropriate position and the imaging device that appears in an optical image when the imaging device at the appropriate position is captured by an optical camera according to the first embodiment;

FIG. 7 illustrates an example of an optical image displayed on an imaging support screen of a display part and a framing image superposed on the optical image according to the first embodiment;

FIG. 8 illustrates an example of the optical image displayed on the imaging support screen of the display part according to the first embodiment, wherein the optical image shows a subject and the imaging device captured by the optical camera;

FIG. 9 illustrates an example of the imaging support screen displayed on the display part when the imaging device in the optical image matches with the framing image according to the first embodiment;

FIG. 10 illustrates an example of matching information displayed on the imaging support system screen of the display part according to the first embodiment;

FIG. 11 illustrates an example of non-matching information displayed on the imaging support system screen of the display part according to the first embodiment;

FIG. 12 illustrates an example of recognition-failure information displayed on the imaging support system screen of the display part according to the first embodiment;

FIG. 13 is a flowchart as an example of operations of the imaging support system server that determines whether the relative position between the radiation source and the imaging device is appropriate before radiographic imaging according to the second embodiment; and

FIG. 14 illustrates an example of the configuration of the imaging support system screen for selecting an SID and so forth displayed on the display part according to the second embodiment.

### DETAILED DESCRIPTION

A radiographic imaging support apparatus, a radiographic imaging support system, a radiographic imaging support method, and a program according to preferred embodiments of the present disclosure will be described in detail with reference to the accompanying drawings. However, the scope of the invention is not limited to the disclosed embodiments.

### Configuration Example of Radiographic Imaging Support System 100

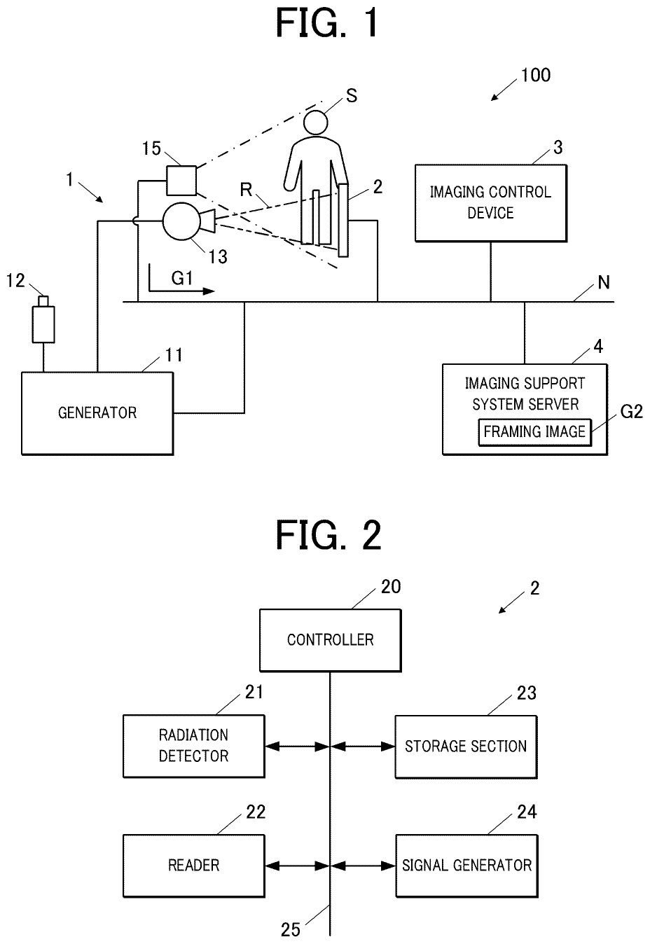

FIG. 1 is a diagram illustrating a schematic configuration of a radiographic imaging support system 100 according to the first embodiment. The radiographic imaging support system 100 includes an irradiation device 1, a radiographic imaging device 2, an imaging control device 3, and an imaging support system server 4. Hereinafter, the radiographic imaging device 2 may be referred to as the imaging device 2. The imaging support system server 4 corresponds to an example of a radiographic imaging support system. The devices/apparatuses constituting the radiographic imaging support system 100 conform to a DICOM standard, and communications between the devices are performed according to the DICOM. DICOM is an abbreviation for Digital Image and Communications in Medicine. Communications between the devices may not be performed according to the DICOM standard. For example, files may be exchanged between the devices by using the FTP. FTP is an abbreviation for File Transfer Protocol.

The irradiation device 1, the imaging device 2, the imaging control device 3, and the imaging support system server 4 are communicably connected to each other via a network N. Examples of the network N include a LAN, a WAN, and the Internet. LAN is an abbreviation for Local Area Network. WAN is an abbreviation for Wide Area Network. If the devices are installed in a medical facility, a LAN can be used, for example. The communication method of the network N may be wired communications or wireless communications.

The irradiation device 1 includes a generator 11, an irradiation instruction switch 12, and a radiation source 13. In response to the irradiation instruction switch 12 being operated, the generator 11 applies voltage to the radiation source 13 having, for example, a tube, based on preset imaging conditions. When voltage is applied by the generator 11, the radiation source 13 generates radiation R having a dose corresponding to the applied voltage. The radiation R is, for example, X-rays. The generator 11 and the radiation source 13 may include an operation receiver that receives inputs of irradiation conditions and a display part. The irradiation device 1 generates the radiation R in a mode corresponding to the type of image to be obtained, such as still image or moving image. The irradiation device 1 may perform processing of automatically recognizing and trimming the irradiation field of the radiation source 13 depending on the imaging part, for example. A radiologist may be able to set the size of the irradiation field as desired depending on the imaging part, by adjusting the collimator.

An optical camera 15 is mounted on the irradiation device 1. The optical camera 15 may be housed in a casing that houses the radiation source 13 or may be attached near the radiation source 13 with an attachment mechanism. The optical camera 15 is connected to the imaging support system server +via the network N. The optical camera 15 may not be directly connected to the network N. The optical camera 15 may be connected to the generator 11 via a hub (not illustrated) and connected to the network N via the generator 11. The communication method may be wired communications or wireless communications. The optical axis of the optical camera 15 is parallel to the irradiation axis of the radiation R passing through the center of the irradiation field. The field of view of the optical camera 15 includes, for example, the subject S on the bed and the imaging device 2 arranged near the imaging part of the subject S. That is, the field of view of the optical camera 15 is greater than the irradiation field of the radiation source 13. The optical camera 15 captures an optical image GI that includes the imaging device 2 and the subject S. The optical image GI may be a moving image or a still image. The optical camera 15 transmits optical image data corresponding to the captured optical image G1 to the imaging support system server 4. The optical camera 15 may be operated using an operation part, a display part, or the like of the irradiation device 1 or using the imaging support system server 4.

The imaging device 2 generates digital image data in which the imaging part of the subject S is captured. For example, a portable FPD is used as the imaging device 2. FPD is an abbreviation for Flat Panel Detector. The imaging device 2 transmits the generated image data to the imaging control device 3 via the network N. The imaging device 2 may include a wireless communication unit and a battery to transmit the generated image data to the imaging control device 3 directly or via an access point.

The imaging control device 3 is also called a console and consists of a personal computer, for example. The imaging control device 3 sets imaging conditions for the irradiation device 1, the imaging device 2, and so forth and controls reading operations of the radiographic image captured by the imaging device 2. The imaging control device 3 may automatically set imaging conditions, based on order information transmitted from the RIS, for example. A radiologist or the like may manually set the imaging conditions by operating the operation part. Examples of the imaging conditions include patient conditions related to the subject S, irradiation conditions related to irradiation of the radiation R, and image reading conditions related to image reading of the imaging device 2. The patient conditions include, for example, an imaging site, an imaging direction, and a physique. The irradiation conditions are, for example, a tube voltage (kV), a tube current (mA), an irradiation time (ms), a current-time product (mAs value), an irradiation field size (vertical and horizontal aperture size of a collimator), and a pulse frame rate. The image reading conditions include, for example, a pixel size, an image size, and a frame rate.

The imaging support system server 4 generates a framing image G2. The framing image G2 indicates where the imaging device 2 appears in the optical image G1 when the imaging device 2 is placed at an appropriate position relative to the radiation source 13. The framing image G2 corresponds to an example of a framing. The imaging device 2 is at an appropriate position when (i) the distance between the imaging device 2 and the radiation source 13 is appropriate for the imaging part and (ii) the imaging surface of the imaging device 2 is at an appropriate angle to the emission direction of the radiation R in each examination. Hereinafter, the distance between the radiation source 13 and the imaging device 2 may be referred to as the SID. The imaging support system server +superposes the generated framing image G2 on the optical image G1 that shows the subject S who holds a position and the imaging device 2 arranged near the imaging part of the subject S. Thus, the framing image G2 and the optical image G1 are displayed. The radiologist or the like adjusts the positions of the radiation source 13 and the imaging device 2 by operating the radiation source 13 so that the framing image G2 matches with the outer edge of the imaging device 2 while viewing the imaging support system screen. Therefore, the imaging support system server 4 is preferably arranged near the irradiation device 1. A display part 42, which will be described later, of the imaging support system server 4 may be arranged near the radiation source 13. In the present embodiment, the imaging support system server 4 generates the framing image G2 for adjusting positions of the radiation source 13 and the imaging device 2 and superimposes the framing image G2 on the optical image G1. However, these processes may be performed by the imaging control device 3. That is, the imaging control device 3 may have the function of the imaging support system server 4 according to the present embodiment. For another example, a device other than the imaging control device 3 may be able to execute the function of the imaging support system server 4.

### Configuration Example of Imaging Device 2

FIG. 2 is a block diagram of the imaging device 2 according to the first embodiment. The imaging device 2 includes a controller 20, a radiation detector 21, a reader 22, a storage section 23, and a signal generator 24. The controller 20, the radiation detector 21, the reader 22, the storage section 23, and the signal generator 24 are connected by wires, such as a bus 25. The controller 20 and other components constituting the imaging device 2 receive predetermined power from the irradiation device 1 or the like via a communication cable (not shown), for example.

The controller 20 includes a processor, such as a CPU or a GPU, and a memory, such as a RAM, for example. CPU is an abbreviation for Central Processing Unit. GPU is an abbreviation for Graphics Processing Unit. RAM is an abbreviation for Random Access Memory. The controller 20 executes a program P stored in the memory or the storage section 23 to perform processing including control related to radiographic imaging, for example.

The radiation detector 21 includes at least radiation detection elements and a substrate. The radiation detection elements directly or indirectly generate charges corresponding to the dose of radiation received from the outside. On the substrate, multiple pixels including switch elements are two dimensionally arranged. The switch elements can switch between conduction and non-conduction between the radiation detection elements and the wiring.

The reader 22 reads out signal values corresponding to the amount of charges accumulated in the radiation detection elements and, based on the read signal values, generates image data of a radiographic image.

The storage section 23 includes, for example, at least one storage module, such as an HDD, an SSD, a ROM, and a RAM. HDD is an abbreviation of Hard Disk Drive. SSD is an abbreviation for Solid State Drive. ROM is an abbreviation of Read Only Memory. The storage section 23 stores, for example, an application program and image data of a radiographic image read by the reader 22.

The signal generator 24 includes a gyro sensor that detects a posture and an angle of the imaging device 2. The signal generator 24 transmits wireless signals including information on the distance from the radiation source 13 and the angle of the imaging device 2. The wireless signals are, for example, signals using an ultra wide band (UWB).

As a method for calculating the distance between the radiation source 13 and the imaging device 2, for example, the positioning technology can be used. The signal generator 24 is mainly used to adjust the positions of the radiation source 13 and the imaging device 2 when the imaging device 2 is not shown in the optical image G1 captured by the optical camera 15.

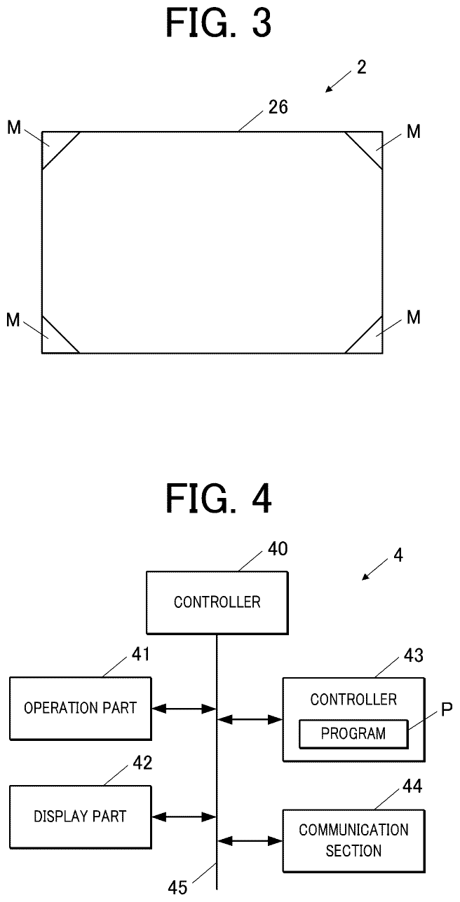

FIG. 3 is an example of a marker M printed on the imaging device 2 according to the first embodiment. The imaging device 2 includes a casing 26 having a rectangular shape in plan view. The marker M is printed at each corner of the casing 26. The marker M is used in calculating the SID and the angle of the imaging device 2 to the irradiation direction of the radiation R. For example, the marker M has a triangular shape to correspond to the shape of the corner of the imaging device 2. The imaging support system server 4 calculates the SID and the angle, based on the size and the shape of the marker M of the imaging device 2 appearing in the optical image G1 captured by the optical camera 15. For example, when the size of the marker M is greater than a reference marker determined beforehand for each imaging region, it means that the distance between the imaging device 2 and the radiation source 13 is short. When the shape of the marker M is distorted compared to the shape of the reference marker, it means that the imaging device 2 is inclined with respect to the irradiation direction of the radiation R. In FIG. 3, the markers M are provided at the four corners of the imaging device 2 to improve the detection accuracy of the imaging device 2. However, the markers M may not be provided at four places. The marker portions M may be provided at five or more positions of the imaging device 2 or may be provided at less than four positions. Further, the shape of the marker M is not limited to the triangular shape shown in FIG. 3. The marker M may have a rectangular shape, a circular shape, or the like.

### Configuration example of Imaging Support System Server 4

FIG. 4 is a block diagram of the imaging support system server 4 according to the first embodiment. The imaging support system server 4 includes a controller 40, an operation part 41, a display part 42, a storage section 43, and a communication section 44. The controller 40, the operation part 41, the display part 42, the storage section 43, and the communication section 44 are communicably connected via wires, such as a bus 45, for example.

The controller 40 includes a processor, such as a CPU, and a memory, such as a RAM, for example. By executing the program P stored in the memory or the storage section 43, for example, the controller 40 displays information for assisting adjustment of the radiation source 13 and the imaging device 2 to their appropriate positions. The controller 40 may include an electronic circuit, such as an ASIC or an FPGA. ASIC is an abbreviation of Application Specific Integrated Circuit. FPGA is an abbreviation for Field Programmable Gate Array.

The controller 40 functions as an optical image acquisition unit, an imaging range determination unit, a detection unit, and a notification unit. By executing the program P stored in the storage section 43, the controller 40 performs functions of the optical image acquisition unit, the imaging range determination unit, the detection unit, and the notification unit. The optical image acquisition unit acquires the optical image G1 that shows the portable imaging device 2 and the subject S. The imaging range determination unit determines the imaging range, based on the information related to the examination of the subject S. The imaging range is the framing image G2 indicating the imaging device 2 and is an irradiation range by the radiation source 13 of the irradiation device 1. The imaging range determination unit determines the framing image G2, based on the optical image GI of the imaging device 2 acquired beforehand by the optical image acquisition unit and the examination information (imaging conditions). The examination information includes SID information, panel information, and irradiation field information. The detection unit detects a position of the imaging device 2. To be specific, the detection unit detects the size, shape, and the like of the marker M of the imaging device 2 by acquiring the marker M provided on the casing 26 of the imaging device 2 appearing in the optical image G1. Based on the size, the shape, and so forth of the detected marker M, the detection unit detects the SID and the angle of the imaging device 2 to the irradiation direction of the radiation R. Based on the detection result, when the position of the imaging device 2 matches with the framing image G2, the notification unit outputs information indicating that the position of the imaging device 2 matches with the framing image G2 to the display part 42.

The operation part 41 receives instructions corresponding to various types of input operations from the user, converts the received instructions into operation signals, and outputs the operation signals to the controller 40. The operation part 41 includes, for example, a mouse, a keyboard, a switch, and a button. The operation part 41 may be, for example, a touch screen integrally combined with a display or may be a user interface, such as a microphone that receives voice input.

The display part 42 displays a GUI for receiving various input operations from the user, the optical image G1 acquired by the optical camera 15, and so forth. The display part 42 is, for example, a display such as a liquid crystal display or an organic EL display. To be specific, the display part 42 displays (i) information for aligning the position of the imaging device 2 in the optical image G1 captured by the optical camera 15 and the framing image G2 generated by the controller 40 and (ii) information for aligning the position of the imaging device 2 in the optical image G1 and the irradiation range of the radiation source 13 of the irradiation device 1. The information for aligning the positions is the imaging support system screen that displays the optical image G1, the framing image G2 superposed and displayed in the optical image G1, and information on whether the framing image G2 matches with the position of the imaging device 2. For another example, the information for aligning the positions is for aligning the position of the imaging device 2 in the optical image G1 and the irradiation range of the radiation source 13 of the irradiation device 1. In the present embodiment, the display part 42 is included in the imaging support system server 4. However, the display part 42 is not limited to the configuration illustrated in FIG. 4. For example, the imaging support system screen that shows the above-described optical image G1, framing image G2, and so forth may be displayed on the display part of the imaging control device 3 or the display part of the radiation source 13 of the irradiation device 1 installed in each imaging room. Further, a tablet terminal may be wirelessly connected to the imaging support system server 4, and the imaging support system screen may be displayed on the display of the tablet terminal. With the tablet terminal, the user is allowed to operate the radiation source 13 at a position closer to the radiation source 13 while viewing the imaging support system screen.

The storage section 43 stores, for example, a system program, an application program, and various types of data. Specifically, the storage section 43 stores the program P for displaying information for supporting the adjustment of the radiation source 13 and the imaging device 2 to their appropriate positions. The storage section 43 includes, for example, a storage module, such as an HDD, an SSD, a ROM, and a RAM.

The communication section 44 includes, for example, a communication module including an NIC, a receiver, and a transmitter. The communication section 44 sends and receives various signals and data to and from the irradiation device 1, the imaging device 2, the imaging control device 3, and the like via the network N. In the present embodiment, the communication section 44 also functions as a receiver that receives a wireless signal transmitted from the signal generator 24 of the imaging device 2.

### Operation Example of Imaging Support System Server 4

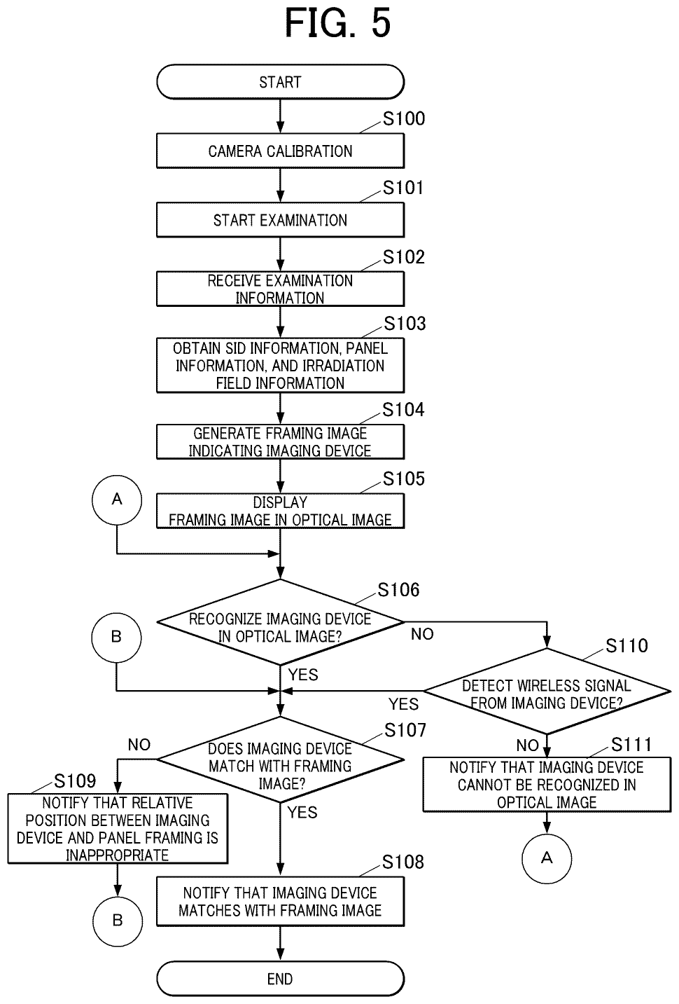

FIG. 5 is a flowchart as an example of operations of the imaging support system server 4 that determines whether the relative position between the radiation source 13 and the imaging device 2 is appropriate before performing radiographic imaging according to the first embodiment. The controller 40 performs the following processing including an acquisition step, a determination step, and a display step by executing the program P.

The controller 40 executes camera calibration before starting the examination through radiographic imaging (step S100). Specifically, the controller 40 acquires camera information of the optical camera 15 mounted on the irradiation device 1 from the imaging control device 3, the cooperating irradiation device 1, and so forth. The camera information includes, for example, various information on the resolution, the image sensor size, and the focal length of the optical camera 15.

In the camera calibration, the optical camera 15 images the imaging device 2 disposed at a predetermined position. The predetermined position corresponds to the SID of 100 cm, for example. As the imaging device 2, an imaging device having a known panel size is used, such as 10×12 inches, 14×17 inches, or 17×17 inches. The controller 40 acquires the optical image G1 captured by the optical camera 15 that shows the imaging device 2. The controller 40 acquires the number of pixels along the longer and shorter sides of the imaging device 2 in the optical image G1. The controller 40 calculates the length per pixel, based on (i) the number of pixels of the longer side and the shorter side of the imaging device 2 in the optical image G1 and (ii) the panel size of the imaging device 2 used for imaging. The panel size information of the imaging device 2 can be acquired from, for example, the imaging device 2 or the imaging control device 3.

After the camera calibration, the radiography examination is started (step S101). Specifically, at the imaging control device 3, the radiological technician or the like selects a predetermined examination, based on the order information transmitted from the RIS or the like. The imaging control device 3 sets imaging conditions corresponding to the selected examination to the imaging device 2 and the irradiation device 1. The radiologist or the like may set imaging conditions by operating the operation part of the irradiation device 1 or the imaging control device 3, for example.

The controller 40 receives examination information from the imaging device 2, the irradiation device 1, the imaging control device 3, and so forth (step S102). The examination information includes, for example, SID information, panel information, irradiation field information, and information on the imaging part. For the SID information, a table in which imaging parts and SIDs are associated with each other beforehand may be prepared, and an SID corresponding to the obtained imaging part may be obtained from the table. Part of the examination information may be acquired from order information transmitted from the RIS.

The controller 40 acquires SID information, panel information, and irradiation field information from the examination information (step S103). As the SID information, an appropriate value is set for each imaging part. For example, when the imaging part is the front of the knee joint, the SID is the 100 cm. The panel information is the panel size of the imaging device 2, such as 10×12 inches, 14×17 inches, or 17×17 inches. For example, when the imaging part is the front of the knee joint, the panel information is 10×12 inches. The irradiation field information is information indicating the range of the radiation R emitted from the radiation source 13. The range of the irradiation field varies depending on the imaging part, for example. Specifically, when the body part to be imaged is a chest, the body part to be imaged is relatively large. In this case, the irradiation field is set to substantially the same range as the panel size of the imaging device 2. When the imaging part is a knee or an elbow, the imaging part is relatively small. In this case, the irradiation field is smaller than the panel size of the imaging device 2. If the irradiation field information is set by default when an examination is selected, the irradiation field information is included in the examination information. On the other hand, when a radiologist or the like manually narrows down the irradiation field of the radiation source 13, the irradiation field information is directly output from the imaging device 2.

The controller 40 generates the framing image G2 that indicates the imaging device 2 and that is to be displayed in the optical image G1 (step S104). The framing image G2 shows the imaging device 2 that is supposed to appear in the optical image G1 when the imaging device 2 is placed at an appropriate position with respect to the radiation source 13.

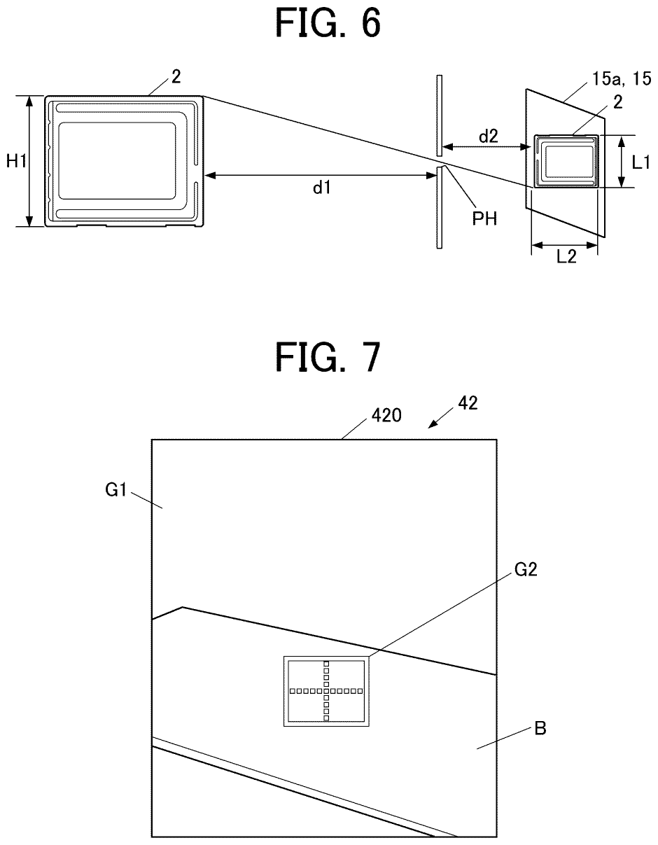

FIG. 6 illustrates the relation in dimensions between the imaging device 2 arranged at an appropriate position and the imaging device 2 that appears in the optical image G1 when the imaging device 2 at the appropriate position is captured by the optical camera 15 according to the first embodiment. In FIG. 6, H1 is the short side of the actual imaging device 2 (hereinafter referred to as the short side H1). Further, d1 is the actual distance (SID) between the image sensor 15a of the optical camera 15 and the imaging device 2 (hereinafter referred to as distance d1). Further, L1 is the short side of the imaging device 2 appearing on the image sensor 15a of the optical camera 15 (hereinafter referred to as the short side L1); and L2 is the long side thereof (hereinafter referred to the long side L2). Further, d2 is the focal distance from the image sensor 15a of the optical camera 15 to the pin hole PH (hereinafter referred to as the focal distance d2).

The short side H1 of the imaging device 2 can be acquired from the panel size of the panel information. The distance d1 can be acquired from the SID information. The focal distance d2 can be acquired as the number of pixels from design information of the optical camera 15. Based on the acquired short side H1, distance d1, and focal distance d2 of the imaging device 2, the controller 40 calculates the number of pixels of the short side L1 of the imaging device 2 appearing on the image sensor 15a. The controller 40 calculates the length of the short side L1 of the imaging device 2 appearing on the image sensor 15a, based on the length per pixel calculated in the camera calibration. The controller 40 calculates the length of the long side L2 of the imaging device 2 appearing on the image sensor 15a by the same method for the short side L1 of the imaging device 2. In this way, the controller 40 generates the framing image G2 that represents the outer edge of the imaging device 2 and that is to be displayed in the optical image G1, based on the calculated short side L1 and long side L2 of the imaging device 2. In the present embodiment, the framing image G2 is generated after the imaging conditions are set as an example. However, the order of these processes may be reversed, or these processes may be performed at the same timing.

The controller 40 superimposes and displays the generated framing image G2 indicating the imaging device 2 on the optical image G1 captured by the optical camera 15 (step S105). FIG. 7 illustrates an example of the optical image G1 displayed on the imaging support system screen 420 of the display part 42 and the framing image G2 superposed on the optical image G1 according to the first embodiment. The imaging support system screen 420 displays the optical image G1 captured by the optical camera 15. In the optical image G1, the framing image G2 indicating the imaging device 2 is superimposed and displayed. Herein, the optical camera 15 is attached at a fixed position with respect to the radiation source 13. Therefore, the center of the irradiation field in the optical image G1 is at a fixed position, and the center of the framing image G2 displayed in the optical image G1 coincides with the center of the irradiation field.

Next, the radiologist guides the patient to the position corresponding to the imaging part and the imaging direction. The radiologist adjusts the positions of the imaging device 2, the patient, and the radiation source 13 so that the imaging part of the patient is within the irradiation field. For example, when the imaging part is the hip joint, the radiologist causes the patient to lie down on the bed B (supine position) and arranges the imaging device 2 to face the knee joint part.

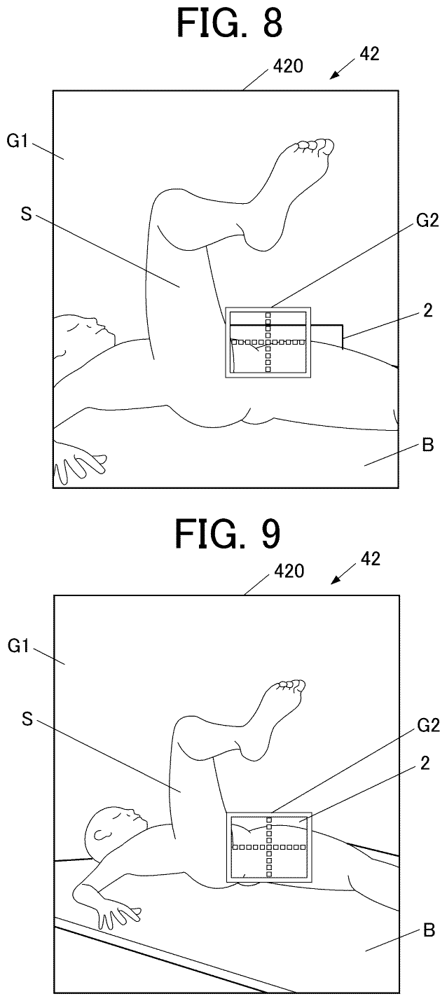

In this state, the optical camera 15 images the subject S and the imaging device 2 arranged at the imaging position. FIG. 8 illustrates an example of the optical image G1 displayed on the imaging support system screen 420 of the display part 42 according to the first embodiment, wherein the optical image G1 shows the subject S and the imaging device 2 captured by the optical camera 15. In the optical image G1, the framing image G2 is shown in addition to the actual subject S and the imaging device 2. The framing image G2 indicates the imaging device 2 that appears in the optical image G1 when the imaging device 2 is arranged at an appropriate position. In FIG. 8, the imaging device 2 is shifted from an appropriate position, so that the imaging device 2 does not match with the framing image G2. The timing of capturing the optical image G1 of the subject S may be a timing before the framing image G2 is displayed.

The controller 40 determines whether the imaging device 2 is recognized in the optical image G1 captured by the optical camera 15 (S106). As the method of recognizing the image of the imaging device 2, machine learning or deep learning as a type of machine learning can be used, for example. As other image recognition methods, pattern matching or other known techniques can be used, for example. When determining that the imaging device 2 is recognized in the optical image G1, the controller 40 proceeds to step S107. Herein, on the imaging device 2 in the optical image G1, the controller 40 may superpose an image (hereinafter referred to as a first similar image) that makes it easier to recognize the imaging device 2 in the optical image G1. The first similar image may be, for example, an image of a frame schematically representing the imaging device 2.

The controller 40 determines whether the imaging device 2 appearing in the optical image G1 matches with the framing image G2 superimposed and displayed on the optical image G1 (step S107). Whether the imaging device 2 in the optical image G1 matches with the framing image G2 can be determined, for example, based on the size and shape of the marker M of the imaging device 2 appearing in the optical image G1. The marker M of the imaging device 2 can be detected by the above-described image recognition technology. At least one marker M of the imaging device 2 is detected.

The controller 40 determines whether the size and the shape of the detected marker M match with the size and shape of the reference marker set in advance. The reference marker is, for example, the marker of the imaging device 2 that appears in the optical image G1 when the imaging device 2 is arranged at an appropriate position for each imaging part. The reference marker is stored beforehand. If the size and shape of the detected marker match with the size and shape of the reference marker, the controller 40 determines that the relative position between the radiation source 13 and the imaging device 2 is appropriate. In other words, the controller 40 determines that the SID is appropriate and that the angle of the imaging device 2 with respect to the radiation source 13 is also appropriate. The controller 40 may determine that the relative position between the radiation source 13 and the imaging device 2 is appropriate when the outer edge of the imaging device 2 appearing in the optical image G1 overlaps with the framing image G2. When the first similar image is superposed and displayed on the imaging device 2 in the optical image G1, the controller 40 may determine whether the imaging device 2 in the optical image G1 matches with the framing image G2, based on whether the framing image G2 matches with the first similar image. Herein, the state where the outer edge of the imaging device 2 appearing in the optical image G1 overlaps with the framing image G2 includes a state where the outer edge of the imaging device 2 appearing in the optical image G1 does not perfectly match with the framing image G2 on a pixel basis. To be specific, the controller 40 sets a pixel value of an allowable deviation range having a predetermined margin with respect to the framing image G2. The controller 40 determines whether the outer edge of the imaging device 2 appearing in the optical image G1 is within the allowable deviation range.

Further, the controller 40 may determine that the imaging device 2 in the optical image G1 matches with the framing image G2 when the following conditions are met. To be specific, the controller 40 may determine that the relative position between the imaging device 2 and the radiation source 13 is appropriate when the imaging device 2 in the optical image G1 matches with the framing image G2 in at least one frame among multiple frames. When the imaging device 2 matches with the framing image G2 in the optical image G1 in N frames among multiple frames of a certain period, the controller 40 may determine that the relative positions of the imaging device 2 and the radiation source 13 are appropriate. Herein, N is a positive integer. The timing at which the controller 40 determines whether the imaging device 2 in the optical image G1 matches with the framing image G2 may be, for example, the timing at which the user presses the irradiation instruction switch 12 by the first stage.

When determining that the imaging device 2 captured in the optical image G1 matches with the framing image G2 shown in the optical image G1, the controller 40 proceeds to step S108. FIG. 9 illustrates an example of the imaging support system screen 420 displayed on the display part 42 when the imaging device 2 in the optical image G1 matches with the framing image G2 according to the first embodiment. When the relative position between the imaging device 2 and the radiation source 13 becomes appropriate, the framing image G2 overlaps with the outer edge of the imaging device 2 in the optical image G1 on the imaging support system screen 420.

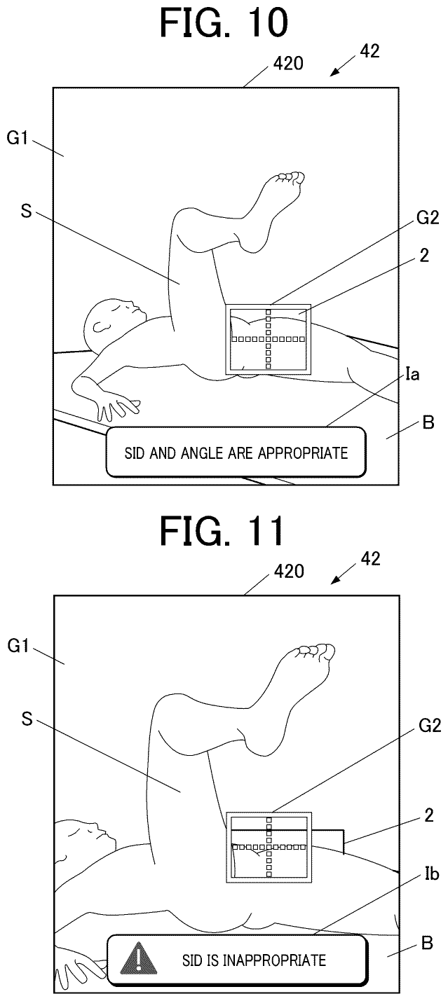

The controller 40 provides the user with matching information Ia indicating that the relative positions of the radiation source 13 and the imaging device 2 are appropriate (step S108). FIG. 10 illustrates an example of the matching information la displayed on the imaging support system screen 420 of the display part 42 according to the first embodiment. The matching information la indicating “SID and angle are appropriate” is displayed on the imaging support system screen 420 of the display part 42. In this case, the radiologist can determine that the relative position between the radiation source 13 and the imaging device 2 is appropriate and can proceed to radiography.

If the controller 40 determines in step S107 that the imaging device 2 appearing in the optical image G1 does not match with the framing image G2 shown in the optical image G1, the controller 40 proceeds to step S109. The controller 40 provides the user with not-matching information Ib indicating that the relative position between the radiation source 13 and the imaging device 2 is inappropriate (step S109). FIG. 11 illustrates an example of the not-matching information Ib displayed on the imaging support system screen 420 of the display part 42 according to the first embodiment. The not-matching information Ib indicating that “SID and angle are inappropriate” is displayed on the imaging support system screen 420 of the display part 42. After displaying the not-matching information Ib on the screen, the controller 40 returns to step S107. In this case, the radiologist adjusts the positions and angles of the radiation sources 13 and so forth so that the framing image G2 matches with the outer edge of the imaging device 2 in the optical image G1 while viewing the framing image G2 and the imaging device 2 in the optical image G1.

Referring back to step S106, if the controller 40 determines that the imaging device 2 cannot be recognized in the optical image G1, the controller 40 proceeds to step S110. Specifically, this is a case where the entire imaging device 2 is hidden by the imaging part or the like of the subject S. In this case, the controller 40 determines whether the imaging device 2 can be recognized, based on wireless signals from the signal generator 24 housed in the imaging device 2 (step S110). The position of the imaging device 2 hidden by the subject S may be estimated by using a trained model trained by machine learning.

If the controller 40 determines that the imaging device 2 can be recognized in the optical image G1, the controller 40 proceeds to step S107. Herein, the controller 40 determines whether the imaging device 2 appearing in the optical image G1 matches with the framing image G2 shown in the optical image G1. The controller 40 acquires information on the SID and the angle of the imaging device 2, based on the wireless signals received from the imaging device 2. Based on the acquired information, the controller 40 generates an image of a framework schematically representing the actual imaging device 2 (hereinafter referred to as a second similar image). The controller 40 determines whether the relative position between the radiation source 13 and the imaging device 2 is appropriate, based on the framing image G2 in the optical image G1 and the second similar image. In a case where the radiation source 13 is moved at the time of aligning the radiation source 13 and the imaging device 2, the angle of view of the optical camera 15 also changes. Accordingly, the second similar image in the optical image G1 also changes and moves according to the position and size of the imaging device 2.

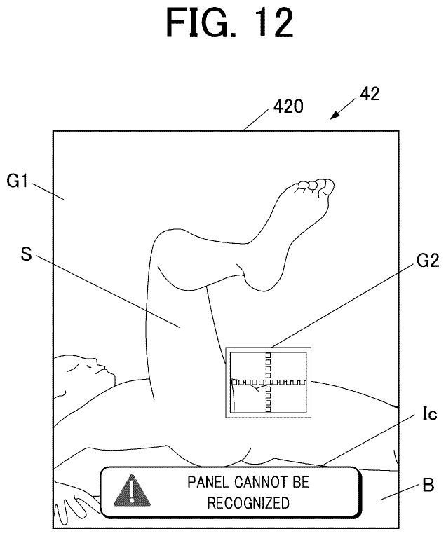

If the controller 40 determines that the imaging device 2 cannot be recognized in the optical image G1, the controller 40 proceeds to step S111. In this case, the controller 40 provides the user with recognition-failure information Ic indicating that the imaging device 2 is not recognizable in the optical image G1 (step S111). FIG. 12 illustrates an example of the recognition-failure information Ic displayed on the imaging support system screen 420 of the display part 42 according to the first embodiment.

On the imaging support system screen 420 of the display part 42, the recognition-failure information Ic saying “panel cannot be recognized” is displayed.

The controller 40 may return to step S106 and determine again whether the imaging device 2 can be recognized in the optical image G1. The radiologist may move the subject S, the imaging device 2, and/or the radiation source 13 so that the imaging device 2 can be recognized in the optical image G1. For another example, radiography can be performed in a state where the imaging device 2 is not recognized in the optical image G1.

According to the first embodiment, the radiologist can check whether the positional relationship between the radiation source 13 and the imaging device 2 is appropriate by looking at the imaging device 2 and the framing image G2 shown in the optical image G1 displayed on the display part +2. Thus, the radiologist is allowed to avoid imaging failure owing to insufficient alignment between the radiation source 13 and the imaging device 2. Accordingly, an appropriate radiographic image can be obtained, and the risk of re-imaging can be reduced.

According to the first embodiment, the size and shape of the marker M of the imaging device 2 is detected to determine whether the SID of the imaging device 2 is appropriate and whether the angle of the imaging device 2 with respect to the radiation is appropriate. Thus, it is possible to accurately check whether the positional relationship between the radiation source 13 and the imaging device 2 is appropriate.

According to the first embodiment, based on the detection result of the marker M of the imaging device 2, the controller 40 displays information on whether the imaging device 2 matches with the framing image G2 in the optical image G1 on the imaging support system screen 420 of the display part 42. Thus, the radiologist can accurately grasp the positional relationship between the radiation source 13 and the imaging device 2 by viewing the imaging support system screen 420. When the not-matching information Ib indicating that the imaging device 2 does not match with the framing image G2 in the optical image G1 is displayed, the radiologist can adjust the position of the radiation source 13 while viewing the imaging support system screen 420. Thus, the alignment between the radiation source 13 and the imaging device 2 can be quickly performed accurately.

### Second Embodiment

In the second embodiment, when the examination information cannot be automatically acquired from the irradiation device 1 or the like, the examination information is manually acquired using the imaging support system screen 420 or the like. Hereinafter, components substantially common to those of the first embodiment are denoted by the same reference numerals, and detailed description thereof is omitted.

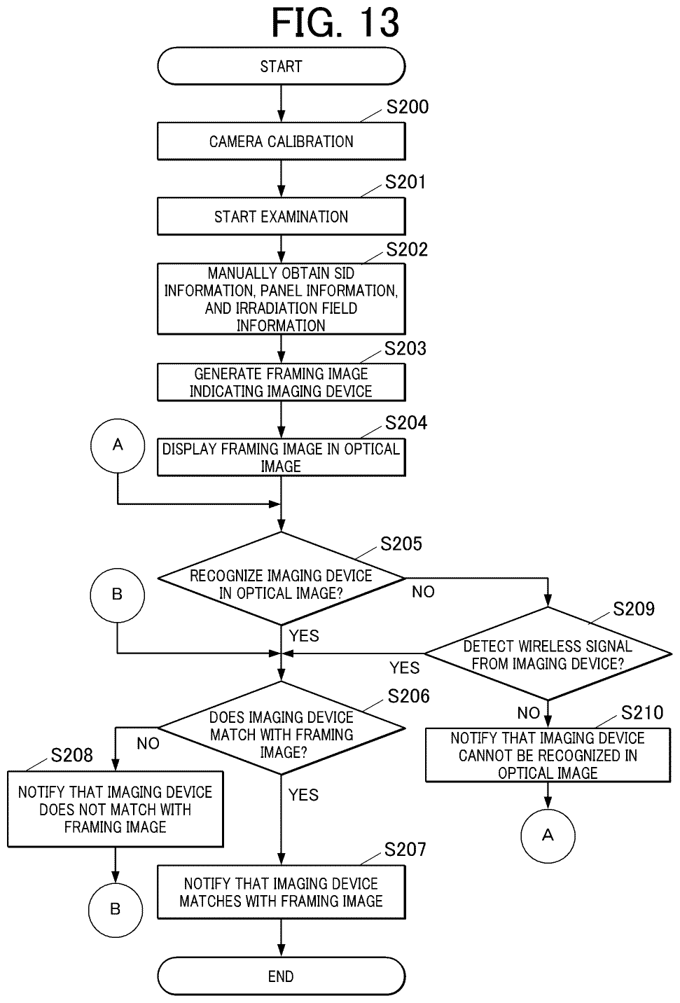

FIG. 13 is a flowchart as an example of operations of the imaging support system server 4 that determines whether the relative position between the radiation source 13 and the imaging device 2 is appropriate before performing radiographic imaging according to the second embodiment.

The controller 40 executes camera calibration before starting the examination through radiographic imaging (step S200). After the camera calibration, the radiography examination is started (step S201).

Herein, depending on the type of the imaging device 2 (e.g., CR), for example, the imaging device 2 may be unable to cooperate with the irradiation device 1 and so forth and unable to acquire examination information from the irradiation device 1. In this case, the controller 40 acquires the SID information, the panel information, and so forth by the selecting operations of the radiologist on the imaging support system screen 420 (step S202).

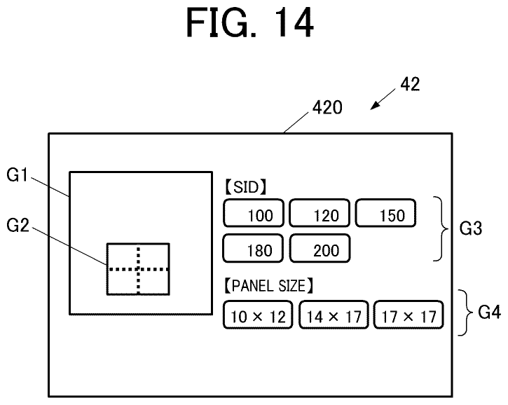

The controller 40 displays a selection image for allowing selections of an SID and a panel size of the imaging device 2 on the imaging support system screen 420 of the display part 42. FIG. 14 illustrates an example of the configuration of the imaging support system screen 420 for selecting an SID and so forth displayed on the display part 42 according to the second embodiment. The imaging support system screen 420 displays an SID selection image G3 for selecting an SID and a panel size selection image G4 for selecting a panel size of the imaging device 2. The SID selection image G3 includes buttons indicating SIDs. The buttons indicating the SIDs are, for example, 100 cm, 120 cm, 150 cm, 180 cm, and 200 cm. The radiologist can select an appropriate SID according to the imaging part and so forth.

The panel size selection image G4 includes multiple panel size buttons. The buttons indicating the panel sizes are, for example, 10×12 inches, 14×17 inches, and 17×17 inches. Herein, the imaging device 2 having a predetermined panel size may be provided by multiple vendors. In this case, the different vendors perform different kinds of camera calibration on the imaging device 2. For example, if the imaging device 2 of 14×17 inches is provided by multiple vendors, there may be multiple buttons of 14×17 inches according to the number of vendors. Thus, the radiologist can select an appropriate panel size according to the imaging part, the vendor, and so forth.

The radiologist may select the selection image by placing and pressing a cursor on the target SID selection image G3 or panel size selection image G4 by manipulating the operation part 41. In a case where the display part 42 is a touch screen, the radiologist may select the selection image by touching the target SID selection image G3 or panel size selection image G4.

The panel size of the imaging device 2 may be obtained with a method using a QR code (registered trademark) or a barcode, for example. For example, an OR code may be attached or printed on the edge portion of the casing 26 of the imaging device 2. The radiologist moves the imaging device 2 to a position readable by the optical camera 15. The optical camera 15 reads the QR code attached to the imaging device 2. The controller 40 acquires information on the panel size of the imaging device 2 stored in the QR code read by the optical camera 15.

In the second embodiment, the framing image G2 to be displayed in the optical image G1 is generated, based on the SID information and the panel information selected by the radiologist on the imaging support system screen 420. The controller 40 determines whether the imaging device 2 appearing in the optical image G1 matches with the framing image G2 and displays a message corresponding to the determination result on the display part 42. Since the process of steps S205 to S210 is the same as the process of steps S106 to S111 of the first embodiment, the description of each step is omitted.

According to the second embodiment, the same operational effects as the above-described first embodiment can be obtained. According to the second embodiment, even if the examination information cannot be automatically acquired from the irradiation device 1 or the like, the radiologist can manually input the examination information on the imaging support system screen 420. Accordingly, even when a CR or the like is used as the imaging device 2, it is possible to check whether the positional relationship between the radiation source 13 and the imaging device 2 is appropriate.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art. The scope of the present invention should be interpreted by terms of the appended claims.

In the above embodiment, the imaging device 2 appearing in the optical image G1 when the imaging device 2 is set at an appropriate position is represented by the framing image G2, and the framing image G2 represents the outer edge of the imaging device 2. However, the present invention is not limited to this. For example, the imaging device 2 arranged to have an SID and an angle corresponding to the imaging part may be expressed by the projection light. In this case, the irradiation device 1 includes a projection unit for projecting the irradiation field (irradiation range) by the radiation source 13. The irradiation field is determined, based on the information on the examination of the subject, and the projection light has the same shape and size as the irradiation field. The projection unit may emit projection light of a special color so that the radiologist can visually recognize the projection light even when the light of the room is off. When the examination starts, the radiologist turns on the projection unit to emit projection light toward the imaging device 2. The radiologist may move the radiation source 13 and so forth so that the projection light coincides with the imaging device 2 using the emitted projection light as a clue. For another example, the radiologist may image the projection light by the optical camera 15 and perform alignment between the projection light and the imaging device 2 as in the first and second embodiments.

1. A radiographic imaging support apparatus comprising a hardware processor and a display, wherein:
the hardware processor obtains an optical image that shows a portable radiographic imaging device and a subject,
the hardware processor determines an imaging range, based on examination information related to an examination of the subject, and
the display displays information for aligning a position of the radiographic imaging device in the obtained optical image and a position of the determined imaging range.

2. The radiographic imaging support apparatus according to claim 1, wherein the imaging range is a framing that indicates the radiographic imaging device.

3. The radiographic imaging support apparatus according to claim 1, wherein the hardware processor determines the imaging range, based on an optical image of the radiographic imaging device obtained beforehand and an imaging condition.

4. The radiographic imaging support apparatus according to claim 3, wherein the imaging condition includes (i) information on a distance between the radiographic imaging device and an irradiation device, (ii) information on a size of the radiographic imaging device, and (iii) information on an irradiation field of the irradiation device.

5. The radiographic imaging support apparatus according to claim 1, wherein the imaging range is an irradiation range by an irradiation device.

6. The radiographic imaging support apparatus according to claim 5, wherein the irradiation range is determined, based on (i) information on a distance between the radiographic imaging device and the irradiation device, (ii) information on a size of the radiographic imaging device, and (iii) information on an irradiation field of the irradiation device, the (i) to (iii) being included in the examination information of the subject.

7. The radiographic imaging support apparatus according to claim 2, wherein:
the hardware processor detects a position of the radiographic imaging device, and
the hardware processor provides a notification when the detected position of the radiographic imaging device matches with the framing of the imaging range on a screen of the display.

8. The radiographic imaging support apparatus according to claim 7, wherein the hardware processor detects the position of the radiographic imaging device, based on the obtained optical image.

9. The radiographic imaging support apparatus according to claim 8, wherein the hardware processor detects the position of the radiographic imaging device by obtaining an image of a marker provided on a casing of the radiographic imaging device.

10. The radiographic imaging support apparatus according to claim 9, wherein the hardware processor detects an angle of the radiographic imaging device to an irradiation direction, based on the image of the marker.

11. The radiographic imaging support apparatus according to claim 1, wherein the display is provided near a radiation source of an irradiation device.

12. The radiographic imaging support apparatus according to claim 1, further comprising a projection unit that projects light corresponding to an irradiation range by a radiation source, the irradiation range being determined based on the examination information of the subject.

13. A radiographic imaging support system comprising:
the radiographic imaging support apparatus according to claim 1; and
the portable radiographic imaging device that images the subject.

14. A radiographic imaging support method comprising:
obtaining an optical image that shows a portable radiographic imaging device and a subject;
determining an imaging range, based on examination information related to an examination of the subject; and
displaying information for aligning a position of the radiographic imaging device in the obtained optical image and a position of the determined imaging range.

15. A non-transitory computer-readable storage medium storing a program that causes a computer to:
obtain an optical image that shows a portable radiographic imaging device and a subject,
determine an imaging range, based on examination information related to an examination of the subject, and
display information for aligning a position of the radiographic imaging device in the obtained optical image and a position of the determined imaging range.
